(19) United States
(12) Patent Application Publication
(10) Pub. No.: US 2013/0325049 A1
(43) Pub. Date: Dec. 5, 2013

(54) REPLACEABLE MICRO-SURGICAL INSTRUMENT

(71) Applicant: NATIONAL CENTRAL UNIVERSITY, Jhongli City (TW)

(72) Inventors: Jason Shiang Ching JANG, Jhongli City (TW); Pei-Hua TSAI, Tainan City (TW); Jia-Bin LI, Huatan Township (TW); Pei-Chun WONG, New Taipei City (TW); Chih-Ching HUANG, Kaohsiung City (TW)

(73) Assignee: NATIONAL CENTRAL UNIVERSITY, Jhongli City (TW)

(21) Appl. No.: 13/716,568

(22) Filed: Dec. 17, 2012

(30) Foreign Application Priority Data
May 30, 2012 (TW) 101119317

(51) Int. Cl. A61B 17/00; A61B 17/32; A61B 17/29

(52) U.S. Cl. CPC A61B 17/00234; A61B 17/2909; A61B 17/320016
USPC 606/170; 606/1; 606/205

(57) ABSTRACT

The present invention provides a replaceable micro-surgical instrument which comprises a pull structure and a hollow tube attached to a grip. The hollow tube has a sleeve at one end with a U-type elastic element sliding in. The U-type elastic element includes a spring tension section and two opposite extending arms and the outer edge of each arm is against the inner wall of the sleeve. Two arms could move according to the pull structure to selectively extend out of the sleeve to be opened or pull back into the sleeve to be clip together. Particularly, there is a replaceable micro-surgical element set in the front-end of each arm, so when the micro-surgical element becomes blunt or broken, the doctor could only replace the micro-surgical element without replace a whole new instrument in order to reduce instrument cost, increase replacement rate, and maintain the quality of medical surgery.

### CROSS REFERENCE TO RELATED APPLICATION

This application claims the benefits of the Taiwan Patent Application Serial Number 101119317, filed on May 30, 2012, the subject matter of which is incorporated herein by reference.

### BACKGROUND OF THE INVENTION

1. Field of the Invention

The present invention relates to a replaceable micro-surgical instrument, more particularly, to a surgical instrument having replaceable micro-surgical elements.

2. Description of Related Art

In present medical surgery, micro surgical instruments are often used in the surgery related to chest or abdomen treatment, such as cutting disease tissues using a micro cutting tool. Existing micro surgical instruments are usually a single instrument for a single purpose and therefore cause high depletions. But high price of micro surgical instruments causes low replacement rate and results in unsuccessfully excision of disease tissues or pulling and dragging of healthy organizations, the postoperative healing becomes more difficult. Moreover, if the disease tissues are cancer cells, the cancer cells would possibly spread to healthy tissue during pulling and dragging tissues. If the cutting tool or forceps are not clear, the possibilities of infection after surgery would decrease.

On the other hand, the surgical instruments used in the surgery are generally made of martensitic stainless steel, a crystalline structure, and its elastic limit is less than 0.5%, and thereby the shift of the normal vector of force away from the center axis often causes the breaking of surgical instruments, resulting in surgical inconvenience. Because the minimally invasive surgery usually needs to clip or twist the surgical instruments, the shift of force away from the center axis would cause the breaking of the surgical instruments, resulting in failure of the surgery and even medical disputes.

Hence, it is desirable to develop a micro surgical instrument which has a relatively low price and excellent properties of good fracture resistance, enhanced corrosion resistance, high wear resistance, improved toughness and high hardness, and is advantageous to the operation of chest or abdomen treatment.

### SUMMARY OF THE INVENTION

The present invention provides a replaceable micro-surgical instrument which comprises a grip, a pull structure, and a hollow tube. The hollow tube has a sleeve at one end with a U-type elastic element sliding set in the sleeve and another end of the hollow tube attached to the grip. A wire set within the hollow tube with one end attached to the pull structure and another end attached to the U-type elastic element. The U-type elastic element includes a spring tension section and two arms opposite extending from the spring tension section. The outer edge of each arm is against the inner wall of the sleeve. Therefore, two arms could selectively extend out of the sleeve to be opened or pull back into the sleeve to be clip together. And there is a replaceable micro-surgical element set in the front-end of each arm.

The micro-surgical element and the correspondingly arm of the U-type elastic element could connect to each other using a dovetail joint, a bolt, or other equivalent structure which can be easily assembly or disassembly by hands. Because the micro-surgical element at the front-end of the present invention is replaceable, the doctor could replace the micro-surgical element for forceps or cutting tools according to the need before a surgery. Further, when the micro-surgical element becomes blunt or broken, the doctor could only replace the micro-surgical element without replace a whole new instrument in order to reduce instrument cost, increase replacement rate, and maintain the quality of medical surgery.

The micro-surgical elements mentioned above are metallic glass micro-surgical elements, such as metallic glass forceps tips or metallic glass cutting elements, which are made by a composite material represented by a following formula 1, formula 2 formula 3 or formula 4,

(ZrₐCu_{b}Ni_{c}Al_{d})₁₀₀₋ₓSiₓ, [Formula 1]

wherein 45=<a=<75, 25=<b=<35, 5=<c=<15, 5=<d=<15, 0.1=<x=<10,

(ZrₑCu_{f}Ag_{g}Alₕ)_{100-y}Si_{y}, [Formula 2]

Wherein 35=<e=<55, 35=<f=<55, 5=<g=<15, 5=<h=<15, 0.1=<y=<10,

TiₐCu_{b}Pd_{c}Zr_{d}Ziₑ, [Formula 3]

Wherein 40=<a=<75, 30=<b=<40, 10=<c=<20, 5=<d=<15, 0.05=<e=<2.

Ti_{f}Zr_{g}TaₕSiᵢ [Formula 4]

Wherein 50=<f=<80, 0=<g=<20, 0=<h=<20, 5=<i=<20.

Furthermore, the micro-surgical elements mentioned above can also be metallic glass micro-surgical elements, such as forceps tips or cutting elements coated with a metallic glass thin film. A composition of the metallic glass thin film is represented by a following formula 1, formula 2 formula 3 or formula 4,

(ZrₐCu_{b}Ni_{c}Al_{d})₁₀₀₋ₓSiₓ, [Formula 1]

wherein 45=<a=<75, 25=<b=<35, 5=<c=<15, 5=<d=<15, 0.1=<x=<10,

(ZrₑCu_{f}Ag_{g}Alₕ)_{100-y}Si_{y}, [Formula 2]

Wherein 35=<e=<55, 35=<f=<55, 5=<g=<15, 5=<h=<15, 0.1=<y=<10,

TiₐCu_{b}Pd_{c}Zr_{d}Ziₑ, [Formula 3]

Wherein 40=<a=<75, 30=<b=<40, 10=<c=<20, 5=<d=<15, 0.05=<e=<2.

Ti_{f}Zr_{g}TaₕSiᵢ [Formula 4]

Wherein 50=<f=<80, 0=<g=<20, 0=<h=<20, 5=<i=<20.

“A metallic glass” means that the metal atoms in the amorphous metal have no long-term continuous regular arrangement, but only short term-regular arrangement. In other words, the atomic arrangement in metallic glass is very different from that in metal with long-term continuous regular arrangement. Metallic glass has advantages such as high thermo-stability, high tensile strength, high elasticity, and high impact toughness, so metallic glass is suitable for the manufacture of medical instruments. Hence, the micro-surgical elements for clipping or cutting in the present invention are made by metallic glass or coated with a metallic glass thin film, and therefore the surface roughness can be decreased so the friction force can be reduced. For example, when cutting elements are made by metallic glass or coated with a metallic glass thin film according to the present invention, the sharpness of the cutting elements can be improved and thus is favorable for the recovery of surgery patients (e.g. following microsurgical repair of a nerve). Moreover, the improved sharpness of the cutting elements can completely cut off the disease tissues, avoid pulling and dragging tissues, make postoperative healing easier, and decrease the possibilities of infection caused by non-clean cutting elements of forceps after surgery.

The pull structure mentioned above could comprise a press part and an elastic element. When pressing the press part, the wire would be stretched and the U-type elastic element would slide into the sleeve. Then, releasing the press part, the elastic element would apply a restoring force to the press part to restore the wire and let the U-type elastic element sliding out of the sleeve.

The hollow tube could be a cylindrical tube or a flexible hollow tube and the wire could be a metal rod or a rigid, slender rectangular connector or a flexible wire. Thus, the doctor could replace the micro-surgical element according to the need before a surgery.

The U-type elastic element and the wire could connect to each other using a dovetail joint, a bolt, or other equivalent structure which can be easily assembly or disassembly by hands. Hence, because the U-type elastic element is replaceable, when the U-type elastic element becomes elastic fatigue or needs to replace a different U-type elastic element, the micro-surgical instrument could only replace the U-type elastic element without replace a whole new instrument in order to reduce instrument cost.

### BRIEF DESCRIPTION OF THE DRAWINGS

FIG. 1 is a perspective view of a replaceable micro-surgical instrument according to a first preferred embodiment of the present invention;

FIG. 2 is a perspective view of the replaceable micro-surgical instrument according to the first preferred embodiment of the present invention;

FIG. 3 is a perspective view of a U-type elastic element and forceps tips according to the first preferred embodiment of the present invention;

FIG. 4 is a moving schematic according to the first preferred embodiment of the present invention;

FIG. 5 is a perspective view of a U-type elastic element and forceps tips according to a second preferred embodiment of the present invention; and

FIG. 6 is a perspective view of a replaceable micro-surgical instrument according to a third preferred embodiment of the present invention.

### DETAILED DESCRIPTION OF THE PREFERRED EMBODIMENT

Hereinafter, the present invention will be described in further detail with reference to examples and comparative examples. It is to be understood, however, that these examples are illustrative only and the scope of the present invention is not limited thereto. Those skilled in the art will appreciate that various modifications, additions and substitutions are possible, without departing from the scope and spirit of the invention as disclosed in the accompanying claims.

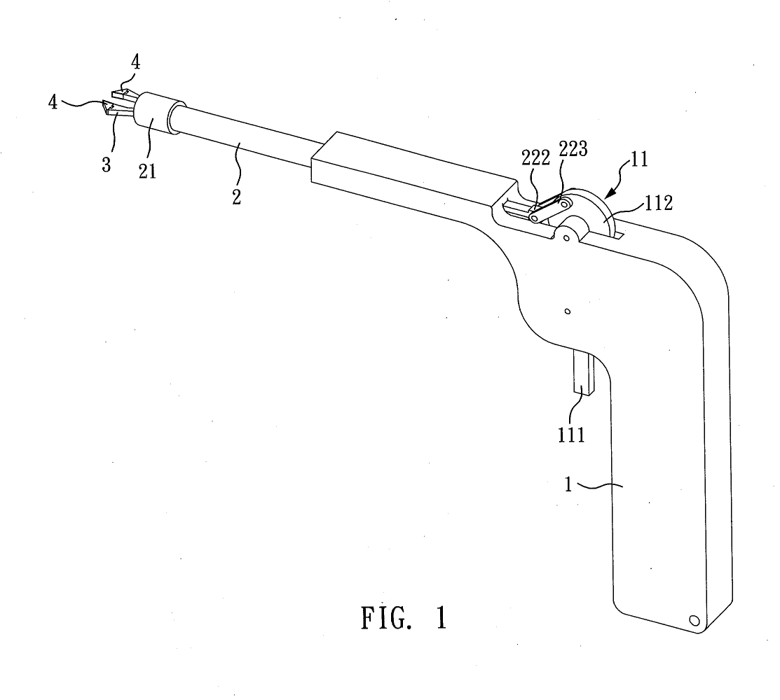

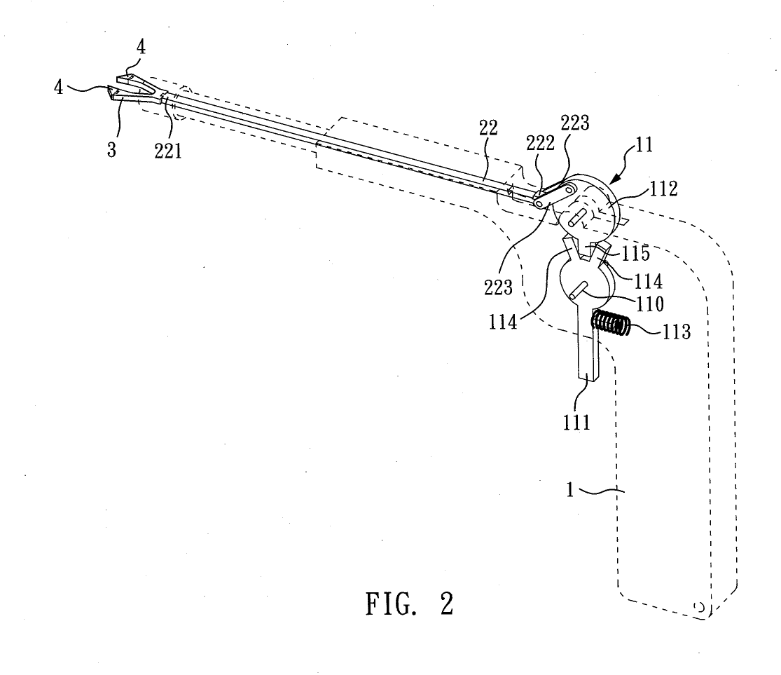

Please referring to FIG. 1 and FIG. 2, FIG. 1 and FIG. 2 both are a perspective view of a replaceable micro-surgical instrument according to a first preferred embodiment of the present invention. The replaceable micro-surgical instrument of the first embodiment of the present invention comprises a grip 1, a pull structure 11, and a hollow tube 2. The hollow tube 2 has a sleeve 21 at one end with a U-type elastic element 3 sliding set in the sleeve 21 and another end of the hollow tube 2 attached to the grip 1. As shown in FIG. 2, a wire 22 set within the hollow tube 2 with one end attached to the pull structure 11 and another end attached to the U-type elastic element 3.

According to FIG. 2, the pull structure 11 is attached to the grip 1 and comprises a press part 111 and an elastic element 113. The elastic element 113 is set between the press part 111 and the grip 1 to apply a restoring force to the press part 111. The press part 111 is attached to the grip 1 by a pivot 110, and is connected to the wire 22 through a rotating disk 112. The press part 111 has two projecting parts 114 and a projecting part 115 of the rotating disk 112 is set between two projecting parts 114 of the press part 111, thus the press part 111 could drive the rotating disk 112 to rotate along the rotation axis. When the elastic element 113 applies the restoring force to the press part 111, the press part 111 would use the restoring force to drive the rotating disk 112 back to the original position.

As shown in FIG. 2, the wire 22 has a first end 221 connected with the U-type elastic element 3 and a second end 222 connected to the non-axis part of the rotating disk 112 of the pull structure 11. In this embodiment, the wire 22 is a metal rod which is connected to the non-axis part of the rotating disk 112 using two parallel bar 223.

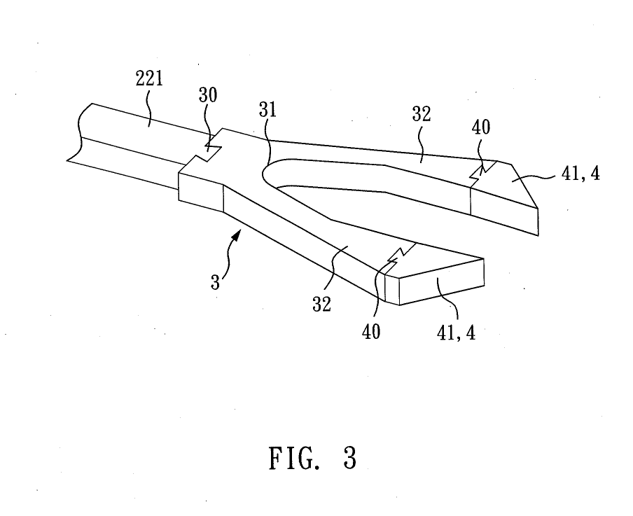

Please referring to FIG. 3, FIG. 3 is a perspective view of the U-type elastic element and forceps tips according to the first preferred embodiment of the present invention. The U-type elastic element 3 includes a spring tension section 31 which is connected to the first end 221 of the wire 22 using a dovetail joint 30. Because the connection between the spring tension section 31 of the U-type elastic element 3 and the wire 22 is a replaceable structure, when the spring tension section 31 of the U-type elastic element 3 becomes elastic fatigue or needs to replace a different U-type elastic element, the micro-surgical instrument could only replace the U-type elastic element 3 without replace a whole new instrument in order to reduce instrument cost.

In addition, the U-type elastic element 3 opposite extends two arms 32 and in the front-end of each arm 32 has a replaceable micro-surgical element 4. In this embodiment, the micro-surgical elements 4 are two metallic glass forceps tips 41 which are connected to the arms 32 using a dovetail joint 40, preferred in tight fit. Because the metallic glass forceps tips 41 connect to the front-end of the arms 32 of the U-type elastic element 3 with a replaceable structure, it could only replace the metallic glass forceps tips 41 when the metallic glass forceps tips 41 becomes blunt or broken without replace a whole new instrument in order to reduce instrument cost, increase replacement rate, and maintain the quality of medical surgery.

The metallic glass forceps tips 41 can be made of metallic glass or forceps tips coated with a metallic glass thin film. A composition of the metallic glass or the metallic glass thin film of this embodiment is represented by a following formula 1, formula 2 formula 3 or formula 4,

(ZrₐCu_{b}Ni_{c}Al_{d})₁₀₀₋ₓSiₓ, [Formula 1]

wherein 45=<a=<75, 25=<b=<35, 5=<c=<15, 5=<d=<15, 0.1=<x=<10,

(ZrₑCu_{f}Ag_{g}Alₕ)_{100-y}Si_{y}, [Formula 2]

Wherein 35=<e=<55, 35=<f=<55, 5=<g=<15, 5=<h=<15, 0.1=<y=<10,

TiₐCu_{b}Pd_{c}Zr_{d}Ziₑ, [Formula 3]

Wherein 40=<a=<75, 30=<b=<40, 10=<c=<20, 5=<d=<15, 0.05=<e=<2.

Ti_{f}Zr_{g}TaₕSiᵢ [Formula 4]

Wherein 50=<f=<80, 0=<g=<20, 0=<h=<20, 5=<i=<20.

Because metallic glass has advantages such as high thermo-stability, high tensile strength, high elasticity, and high impact toughness, it is suitable for the manufacture of medical instruments. Therefore, in the present invention, the metallic glass forceps tips 41 are made of metallic glass or forceps tips coated with a metallic glass thin film. Thus the surface roughness can be decreased so the friction force can be reduced.

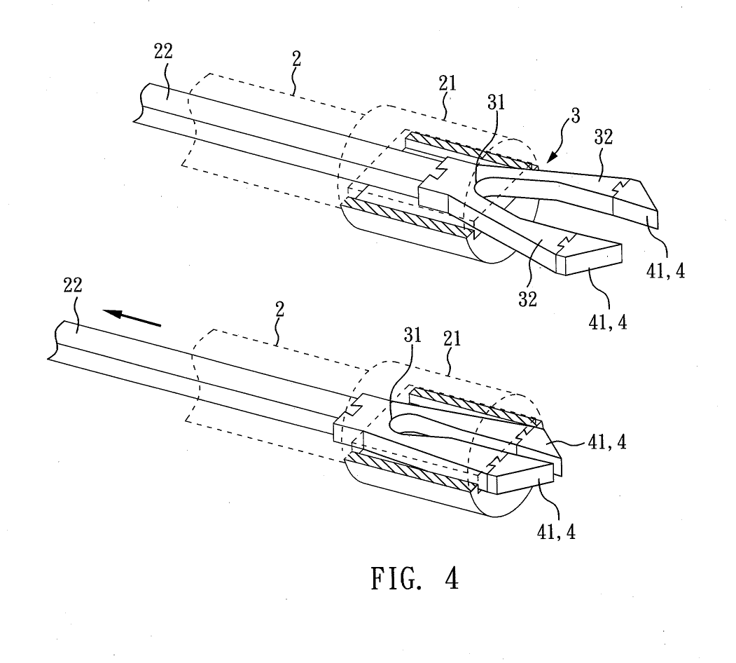

Please referring to FIG. 4 and FIG. 2, FIG. 4 is a moving schematic according to the first preferred embodiment of the present invention. The two arms 32 of the. U-type elastic element 3 which is connected to the wire 22 are against the inner wall of the sleeve 21 and could selectively extend out of the sleeve 21 to be opened or pull back into the sleeve 21 to be clip together. As shown in FIG. 2, when a user presses the press part 111, the press part 111 would rotate along the pivot 110 and drive two projecting parts 114. Two projecting parts 114 would drive the projecting part 115 of the rotating disk 112 to do clockwise rotation along the axis center and the wire 22 which is connected to the rotating disk 112 would be pulled. In the meanwhile, as shown in FIG. 4, when the wire 22 is pulled, the U-type elastic element 3 which is connected to the wire 22 will be pulled back and slide into the sleeve 21. The spring tension section 31 of the U-type elastic element 3 will compress to center due to the inner wall of the sleeve 21 thus the two metallic glass forceps tips 41 will close to each other in order to reach the clip function.

When the user releases the press part 111, the press part 111 will be restored back to the original position because of the restoring force applied by the elastic element 113. Meanwhile, two projecting parts 114 of the press part 111 would drive the projecting part 115 of the rotating disk 112 to do counterclockwise rotation and push the wire 22 which is connected to the rotating disk 112 moving forward. Then, as shown in FIG. 4, the U-type elastic element 3 will be pushed out of the sleeve 21 of the hollow tube 2 and the spring tension section 31 will apply the restoring force to let the arms 32 being opened.

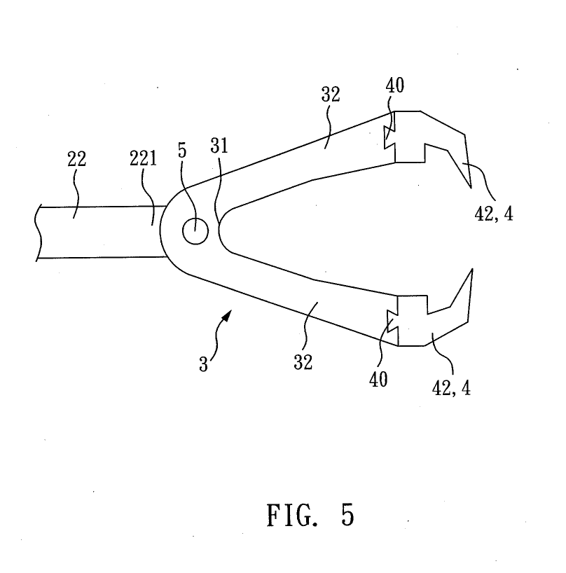

Except metallic glass forceps tips 41 or forceps tips coated with a metallic glass thin film, the micro-surgical elements 4 could be metallic glass knives 42 or knives coated with a metallic glass thin film. Referring to FIG. 5, FIG. 5 is a perspective view of a U-type elastic element and forceps tips according to a second preferred embodiment of the present invention. The difference between the second preferred embodiment and first preferred embodiment is that the micro-surgical elements 4 which are connected to the arms 32 of the U-type elastic element 3 are replaced into the metallic glass knives 42. Similarly, the connection structure between the arms 32 and the metallic glass knives 42 could use a dovetail joint or a bolt. In this embodiment, the connection structure between the arms 32 and the metallic glass knives 42 is using a dovetail joint and preferred in tight fit. Therefore, if the surgery needs a cutting function, the doctor only needs to replace the micro-surgical elements 4 without replace a whole new instrument in order to reduce instrument cost.

The same as mentioned above, the metallic glass knives 42 can be made of metallic glass or knives coated with a metallic glass thin film. A composition of the metallic glass or the metallic glass thin film of this embodiment is represented by a following formula 1, formula 2 formula 3 or formula 4,

(ZrₐCu_{b}Ni_{c}Al_{d})₁₀₀₋ₓSiₓ, [Formula 1]

wherein 45=<a=<75, 25=<b=<35, 5=<c=<15, 5=<d=<15, 0.1=<x=<10,

(ZrₑCu_{f}Ag_{g}Alₕ)_{100-y}Si_{y}, [Formula 2]

Wherein 35=<e=<55, 35=<f=<55, 5=<g=<15, 5=<h=<15, 0.1=<y=<10,

TiₐCu_{b}Pd_{c}Zr_{d}Ziₑ, [Formula 3]

Wherein 40=<a=<75, 30=<b=<40, 10=<c=<20, 5=<d=<15, 0.05=<e=<2.

Ti_{f}Zr_{g}TaₕSiᵢ [Formula 4]

Wherein 50=<f=<80, 0=<g=<20, 0=<h=<20, 5=<i=<20.

Because metallic glass has advantages such as high thermo-stability, high tensile strength, high elasticity, and high impact toughness, it is suitable for the manufacture of medical instruments. Hence, the metallic glass is applied to make the knives or coated on the knives surface, so the surface roughness can be decreased and the friction force can be reduced. Moreover, the improved sharpness of the cutting elements can completely cut off the disease tissues, avoid pulling and dragging tissues, make postoperative healing easier, and decrease the possibilities of infection caused by non-clean cutting elements of forceps after surgery.

In addition, the connection between the spring tension section 31 of the U-type elastic element 3 and the first end of the wire 22 is using a bolt 5, therefore when the spring tension section 31 of the U-type elastic element 3 becomes elastic fatigue or needs to replace a different U-type elastic element, the micro-surgical instrument could only replace the U-type elastic element 3 without replace a whole new instrument in order to reduce instrument cost.

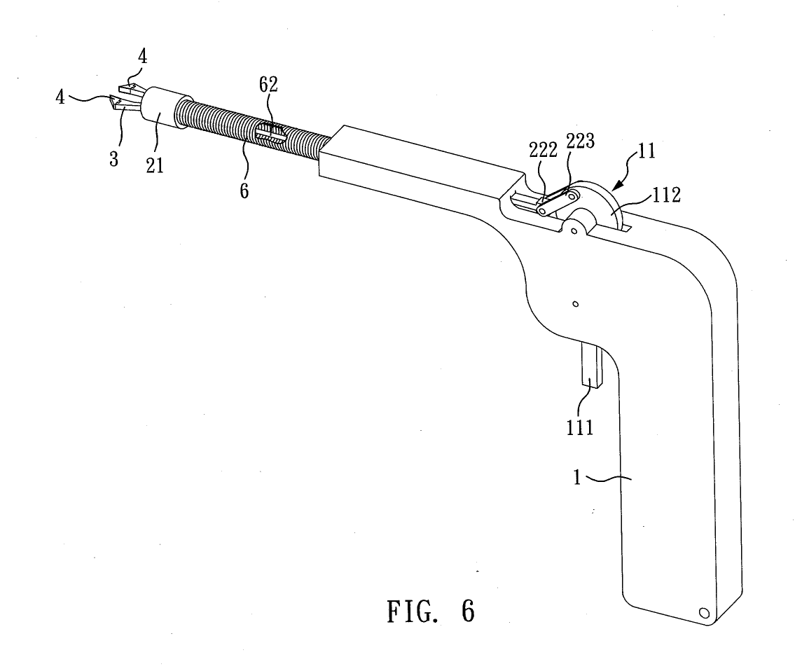

Referring to FIG. 6 which is a perspective view of a replaceable micro-surgical instrument according to a third preferred embodiment of the present invention, the differences between the third preferred embodiment and the first preferred embodiment are that the hollow tube 6 is a flexible hollow tube and the wire 62 is a rigid, slender rectangular connector or a flexible wire in order to replace the micro-surgical element according to the need before a surgery.

Although the present invention has been explained in relation to its preferred embodiment, it is to be understood that many other possible modifications and variations can be made without departing from the spirit and scope of the invention as hereinafter claimed.

1. A replaceable micro-surgical instrument, which comprises:
a grip;
a pull structure attached to the grip; and
a hollow tube having a sleeve at one end with a U-type elastic element sliding set in the sleeve, another end of the hollow tube attached to the grip, and a wire set within the hollow tube with one end attached to the pull structure and another end attached to the U-type elastic element;
wherein the improvement comprises:
the U-type elastic element including a spring tension section and two arms opposite extending from the spring tension section, the outer edge of each arm against inner wall of the sleeve, and front-end of each arm having a replaceable micro-surgical element.

2. The replaceable micro-surgical instrument as claimed in claim 1, wherein the micro-surgical element and the correspondingly arm of the U-type elastic element connect to each other using a dovetail joint.

3. The replaceable micro-surgical instrument as claimed in claim 1, wherein the micro-surgical element and the correspondingly arm of the U-type elastic element connect to each other using a bolt.

4. The replaceable micro-surgical instrument as claimed in claim 1, wherein the micro-surgical element is a metallic glass micro-surgical element.

5. The replaceable micro-surgical instrument as claimed in claim 4, wherein the metallic glass micro-surgical element is a metallic glass forceps tip which is made by a composite material represented by a following formula 1, formula 2 formula 3 or formula 4,
(ZrₐCu_{b}Ni_{c}Al_{d})₁₀₀₋ₓSiₓ, [Formula 1]
wherein 45=<a=<75, 25=<b=<35, 5=<c=<15, 5=<d=<15, 0.1=<x=<10,
(ZrₑCu_{f}Ag_{g}Alₕ)_{100-y}Si_{y}, [Formula 2]
Wherein 35=<e=<55, 35=<f=<55, 5=<g=<15, 5=<h=<15, 0.1=<y=<10,
TiₐCu_{b}Pd_{c}Zr_{d}Ziₑ, [Formula 3]
Wherein 40=<a=<75, 30=<b=<40, 10=<c=<20, 5=<d=<15, 0.05=<e=<2.
Ti_{f}Zr_{g}TaₕSiᵢ [Formula 4]
Wherein 50=<f=<80, 0=<g=<20, 0=<h=<20, 5=<i=<20.

6. The replaceable micro-surgical instrument as claimed in claim 4, wherein the metallic glass micro-surgical element is a metallic glass cutting element which is made by a composite material represented by a following formula 1, formula 2 formula 3 or formula 4,
(ZrₐCu_{b}Ni_{c}Al_{d})₁₀₀₋ₓSiₓ, [Formula 1]
wherein 45=<a=<75, 25=<b=<35, 5=<c=<15, 5=<d=<15, 0.1=<x=<10,
(ZrₑCu_{f}Ag_{g}Alₕ)_{100-y}Si_{y}, [Formula 2]
Wherein 35=<e=<55, 35=<f=<55, 5=<g=<15, 5=<h=<15, 0.1=<y=<10,
TiₐCu_{b}Pd_{c}Zr_{d}Ziₑ, [Formula 3]
Wherein 40=<a=<75, 30=<b=<40, 10=<c=<20, 5=<d=<15, 0.05=<e=<2.
Ti_{f}Zr_{g}TaₕSiᵢ [Formula 4]
Wherein 50=<f=<80, 0=<g=<20, 0=<h=<20, 5=<i=<20.

7. The replaceable micro-surgical instrument as claimed in claim 4, wherein the metallic glass micro-surgical element is a forceps tip coated with a metallic glass thin film, and a composition of the metallic glass thin film is represented by a following formula 1, formula 2 formula 3 or formula 4,
(ZrₐCu_{b}Ni_{c}Al_{d})₁₀₀₋ₓSiₓ, [Formula 1]
wherein 45=<a=<75, 25=<b=<35, 5=<c=<15, 5=<d=<15, 0.1=<x=<10,
(ZrₑCu_{f}Ag_{g}Alₕ)_{100-y}Si_{y}, [Formula 2]
Wherein 35=<e=<55, 35=<f=<55, 5=<g=<15, 5=<h=<15, 0.1=<y=<10,
TiₐCu_{b}Pd_{c}Zr_{d}Ziₑ, [Formula 3]
Wherein 40=<a=<75, 30=<b=<40, 10=<c=<20, 5=<d=<15, 0.05=<e=<2.
Ti_{f}Zr_{g}TaₕSiᵢ [Formula 4]
Wherein 50=<f=<80, 0=<g=<20, 0=<h=<20, 5=<i=<20.

8. The replaceable micro-surgical instrument as claimed in claim 4, wherein the metallic glass micro-surgical element is a cutting element coated with a metallic glass thin film, and a composition of the metallic glass thin film is represented by a following formula 1, formula 2 formula 3 or formula 4,
(ZrₐCu_{b}Ni_{c}Al_{d})₁₀₀₋ₓSiₓ, [Formula 1]
wherein 45=<a=<75, 25=<b=<35, 5=<c=<15, 5=<d=<15, 0.1=<x=<10,
(ZrₑCu_{f}Ag_{g}Alₕ)_{100-y}Si_{y}, [Formula 2]
Wherein 35=<e=<55, 35=<f=<55, 5=<g=<15, 5=<h=<15, 0.1=<y=<10,
TiₐCu_{b}Pd_{c}Zr_{d}Ziₑ, [Formula 3]
Wherein 40=<a=<75, 30=<b=<40, 10=<c=<20, 5=<d=<15, 0.05=<e=<2.
Ti_{f}Zr_{g}TaₕSiᵢ [Formula 4]
Wherein 50=<f=<80, 0=<g=<20, 0=<h=<20, 5=<i=<20.

9. The replaceable micro-surgical instrument as claimed in claim 1, wherein the pull structure comprises a press part and an elastic element, when pressing the press part, the wire would be stretched and the U-type elastic element would slide into the sleeve; then, releasing the press part, the elastic element would apply a restoring force to the press part to restore the wire and let the U-type elastic element sliding out of the sleeve.

10. The replaceable micro-surgical instrument as claimed in claim 1, wherein the hollow tube is a cylindrical tube.

11. The replaceable micro-surgical instrument as claimed in claim 10, wherein the wire is a metal rod.

12. The replaceable micro-surgical instrument as claimed in claim 1, wherein the hollow tube is a flexible hollow tube.

13. The replaceable micro-surgical instrument as claimed in claim 12, wherein the wire is a rigid, slender rectangular connector or a flexible wire.

14. The replaceable micro-surgical instrument as claimed in claim 1, wherein the U-type elastic element and the wire connect to each other using a dovetail joint.

15. The replaceable micro-surgical instrument as claimed in claim 1, wherein the U-type elastic element and the wire connect to each other using a bolt.
